(19) United States
(12) Patent Application Publication
(10) Pub. No.: US 2021/0121226 A1
(43) Pub. Date: Apr. 29, 2021

(54) ARTICULATING MEDICAL GRASPER

(71) Applicant: Medtronic Xomed, Inc., Jacksonville, FL (US)

(72) Inventor: Daniel A. Joseph, Golden, CO (US)

(21) Appl. No.: 17/078,698

(22) Filed: Oct. 23, 2020

Related U.S. Application Data
(60) Provisional application No. 62/927,012, filed on Oct. 28, 2019.

(51) Int. Cl. A61B 18/14; A61B 17/29; A61M 25/01; A61M 25/00; A61M 25/09; A61B 18/00; A61B 34/30; A61B 18/12

(52) U.S. Cl. CPC A61B 18/1445; A61B 17/29; A61M 25/0147; A61M 25/0026; A61B 2018/126; A61B 2017/2927; A61B 2018/00345; A61B 2018/0063; A61B 34/30; A61M 25/09

(57) ABSTRACT

An articulated medical device including, a proximal articulating region, a catheter extending from the proximal articulating region, a distal articulating region, and a plurality of guide wires extending from the proximal to the distal articulating regions and mechanically coupling and transferring movements of the proximal articulating region to the distal articulating region.

### FIELD

The present disclosure relates generally to an articulating medical grasper, and more particularly, to an articulating bipolar vessel sealer

### BACKGROUND

Electrosurgical instruments have become widely used by surgeons for many years. Many electrosurgical instruments are hand-held instruments, e.g., electrosurgical pencil or electrosurgical forceps, which transfer radiofrequency (RF) electrical energy to a tissue site. The electrosurgical energy is returned to the electrosurgical source via a return electrode pad positioned under a patient (i.e., a monopolar system) or a smaller return electrode integrally formed in the forceps and in bodily contact with or immediately adjacent to the surgical site (i.e., a bipolar system). The waveforms produced by the RF source yield a predetermined electrosurgical effect known generally as electrosurgical coagulation and cutting.

To make such devices effective for laparoscopic and robotic surgery, articulating systems have been designed to allow the end effector (e.g., the forceps) to move relative to a shaft to which they are affixed. These articulating systems have taken many forms, and some are better than others. Improvements to articulation systems are desired to allow for greater range of use of surgical tools such as bipolar electrosurgical forceps, that have reduced diameters for laparoscopic surgical procedures.

### SUMMARY

The disclosure is directed to an articulating system that can be employed with a variety of tools to provider greater access and functionality during robotic and laparoscopic surgical procedures.

One aspect of the disclosure is directed to an articulated medical device including: a proximal articulating region including a plurality of articulating hubs, a distal articulating region including a plurality articulating members. The articulated medical device also includes at least one catheter extending between the proximal articulating region and the distal articulating region. The articulated medical device also includes a plurality of pull wires extending from the proximal articulating region to the distal articulating region and mechanically coupling and transferring movements of the proximal articulating region to the distal articulating region, where the movements of the proximal articulating region are amplified to a greater magnitude of movement in the distal articulating region.

Implementations of this aspect of the disclosure may include one or more of the following features. The articulated medical device where the plurality of pull wires are equally tensioned. The articulated medical device including, an inner catheter and an outer catheter, where the outer catheter defines a plurality of lumens configured for receiving the plurality of guide wires. The articulated medical device where the inner catheter defines at least one lumen configured to receive a tool actuator. The articulated medical device further including an end effector operably engaged with the tool actuator. The articulated medical device where the end effector is a forceps. The articulated medical device where the forceps is a bipolar electrosurgical vessel sealer. The articulated medical device further including a tapered hub disposed between the plurality of articulating hubs and the catheter. The articulated medical device where the tapered hub includes an inner portion including a plurality of channels configured for receiving the guide wires. The articulated medical device further including at least one support ring. The articulated medical device where the support ring is configured to receive and secure the plurality of pull wires. The articulated medical device where the distal articulating region includes a plurality of pull wire connectors configured to secure the plurality of pull wires to the distal articulating region. The articulated medical device further including a distal catheter configured to receive an end effector.

A further aspect of the disclosure is directed to an articulated medical device including: a proximal articulating region including a plurality of articulating hubs, a tapered hub, and at least one support ring; at least one catheter extending from the tapered hub including a plurality of lumens formed therein; a distal articulating region including a plurality articulating members, a distal catheter and a plurality of pull wire connectors, where the distal articulating region is configured to mate with the at least one catheter; and a plurality of pull wires secured the at least one support ring and extending from the at least one support ring, through the plurality of articulating hubs, in channels formed in the tapered hub, through the plurality of lumens formed in the at least one catheter, through the plurality of articulating members, and secured to the plurality of pull wire connectors, where the plurality of pull wires are equally tensioned along their length.

Implementations of this aspect of the disclosure may include one or more of the following features. The articulated medical device further including, an inner catheter and an outer catheter, where the outer catheter defines the plurality of lumens configured for receiving the plurality of guide wires. The articulated medical device where the inner catheter defines at least one lumen configured to receive a tool actuator. The articulated medical device further including an end effector operably engaged with the tool actuator. The articulated medical device where the end effector is a forceps. The articulated medical device where the forceps is a bipolar electrosurgical vessel sealer. The articulated medical device where movements of the proximal articulating region are amplified resulting in a greater magnitude of movement in the distal articulating region.

### BRIEF DESCRIPTION OF THE DRAWINGS

Various aspects and features of the disclosure are described hereinbelow with references to the drawings, wherein:

FIG. 1 is a plan view of an articulating medical device in accordance with the disclosure;

FIG. 2 is a cross-sectional view of the articulating medical device of FIG. 1;

FIG. 3 is a plan view of the articulating medical device of FIG. 1 in an articulated position;

FIG. 4 is a cross-sectional view of the articulating medical device in an articulated position of FIG. 3;

FIG. 5 is a rear perspective view of an articulating medical device in accordance with the disclosure;

FIG. 6 is a cross-sectional view of the articulating medical device of FIG. 5;

FIG. 7 is a rear perspective view of the articulating medical device of FIG. 5 with an outer heat shrink layer removed;

FIG. 8 is a rear perspective view of the articulating medical device of FIG. 7 with an outer catheter removed;

FIG. 9 is a rear perspective view of the articulating medical device of FIG. 8 with the pull wires removed;

FIG. 10 is a plan view of a bipolar grasper in accordance with the disclosure.

FIG. 11 is a plan view of an articulating medical device in accordance with the disclosure;

FIG. 12 is a plan view of the articulating medical device of FIG. 11 with the clam shell rendered transparently; and

FIG. 13 is a plan view of an articulating medical device in FIG. 11 with the clam shell rendered transparently and in an unarticulated position.

### DETAILED DESCRIPTION

The disclosure is directed to an articulating medical device that can be employed with a variety of tools to provider greater access and functionality during robotic and laparoscopic surgical procedures.

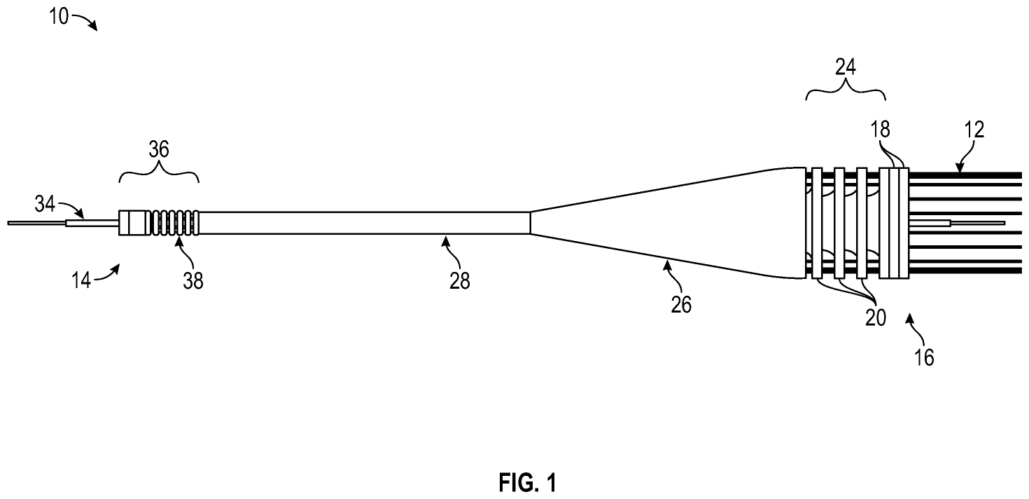

FIG. 1 depicts a profile view of an articulating medical device 10 in accordance with the disclosure. The articulating medical device 10 includes a plurality of pull wires 12. The pull wires 12 extend the length of the medical device 10 and terminate proximate the distal portion 14 of the medical device 10. At a proximal end 16, having a larger diameter, the pull wires 12 extend through two support rings 18. The support rings enable connection of the medical device to a device interface (not shown) such as a handle or a robotic drive mechanism.

Distal of the support rings 18 are a series of nested articulating hubs 20. Each of the articulating hubs 20 and the support rings 18 include orifices 22 through which the pull wires 12 extend. In some embodiments the pull wires may end at and be secured to one of the support rings 18. The nested articulating hubs 20 are received within each other and allow for complete freedom of movement of the proximal portion of the medical device 10. The nested articulating hubs form a proximal articulating portion 24. In accordance with one aspect of the disclosure the nested articulating hubs 20 having an enlarged diameter, as compared to distal aspects of the medical device 10 promotes a greater range of articulation and angle amplification than observed in other systems.

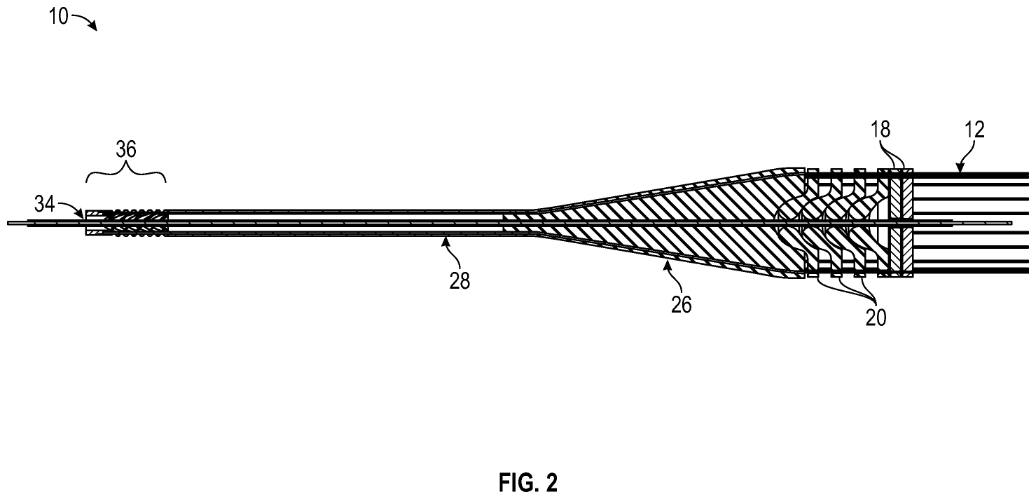

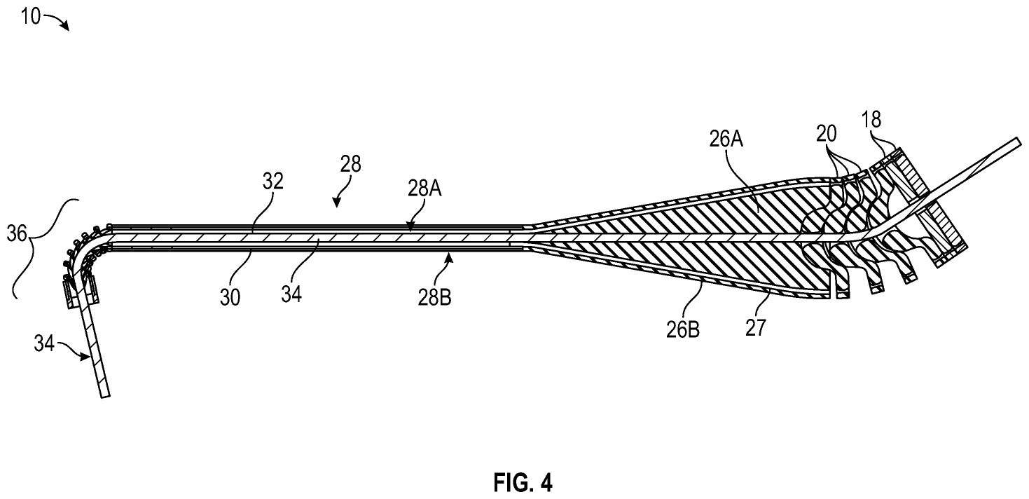

Distal of the nested articulating hubs 20 is a tapered hub 26. The tapered hub 26 provides a transition from the nested articulating hubs 20 to the diameter of the catheters 28 which is distal of the tapered hub. Channels or lumens 27 formed in the periphery of the tapered hub 26 receive the pull wires 12. As depicted in FIGS. 2 and 4 and 8, the tapered hub may be formed of a two part construction with an inner portion 26A having the channels formed therein and an outer cover portion 26B As shown in FIG. 2, lumens 30 which pass through a periphery of the catheters 28, the tapered hub 26, the nested articulating hubs 20, and the support rings 18 receive the pull wires 212 and electrically isolate the pull wires 12 from each other as will be described in more detail below.

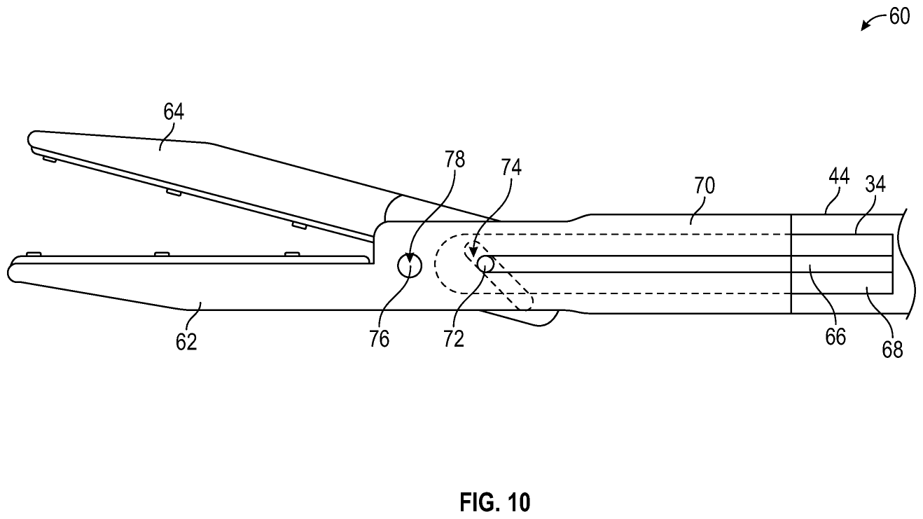

Distal of the tapered hub 26 are a plurality of concentric catheters 28. An inner catheter 28A mates with a distal end of the tapered hub 26. An outer catheter 28B overlaps the inner catheter 28A and includes the lumens 30 formed therein. The lumens 30 in the outer catheter 28B mate with the channels 27 in the tapered hub 26 and allow for the passage of the pull wires 12 therethrough. Those of skill in the art will understand that the plurality of catheters 28 may be separately formed and then fused to one another during the manufacturing process. A larger lumen 32 extends through the inner catheter 28A and mates with a similar lumen formed in the tapered hub 26, the nested articulating hubs 20, and the support rings 18. The larger lumen 32 can receive, for example a tool actuator 34 depicted schematically here. The tool actuator 34 may be for a diagnostic tool such as a biopsy tool. Additionally or alternatively, the tool actuator 34 may be part of a forceps as shown in FIG. 10.

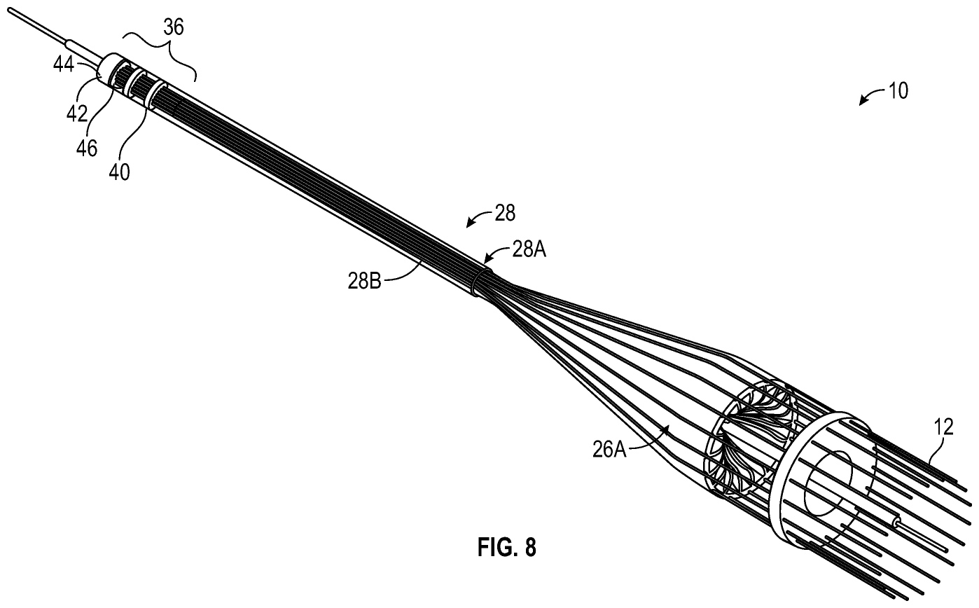

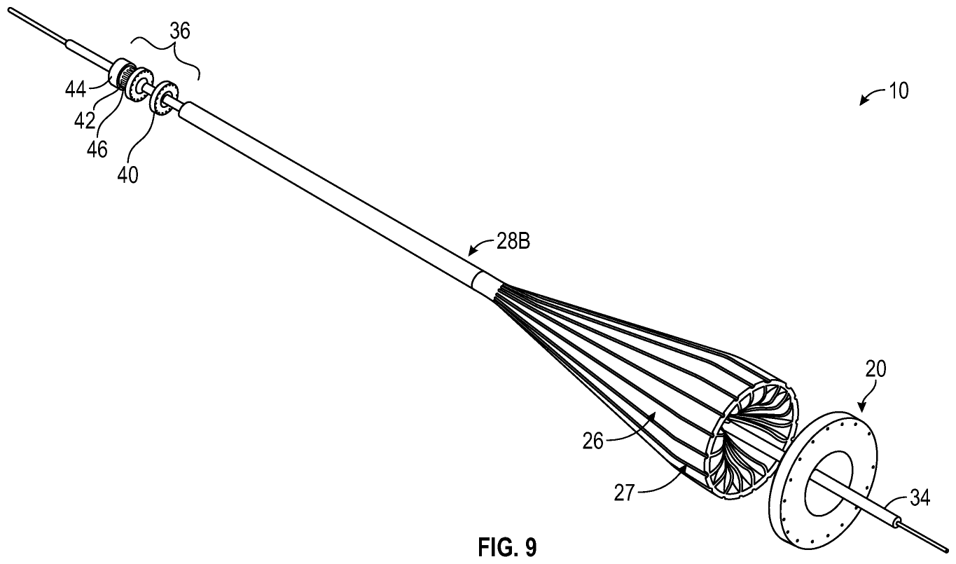

Distal of the catheters 28 is a distal articulating portion 36. As shown the distal articulating portion 36 includes a series of nested articulating members 38 which can move relative to one another to allow the distal portion of the medical device 10 to articulate. The pull wires 12 extend through the nested articulating members 38. The distal articulating portion 36 may also include a series of rings 40 (FIGS. 8 and 9) through which the pull wires 12 traverse. A distal hub 42 forms the distal portion of the medical device 10 and the pull wires 12 are secured to the distal hub 42. At the distal end of the distal hub 42 is a distal catheter 44. The distal hub 42 also includes a pull wire connector 46. The pull wires 12 traverse the nested articulating members 38 and rings 40 and are fastened to the pull wire connector 46.

As depicted in FIG. 2, when the support rings 18, to which the pull wires 12 are attached, are articulated in one direction, causing the nested articulating hubs 20 to articulate to compensate, an opposite reaction is observed at the distal end 14. The distal articulating portion 36 articulates in both an opposite direction and in a greater magnitude of angle traversed than observed at the proximal end 16. As noted above, this differences in magnitude of movement is based on the relative size of the nested articulating hubs 20 and the nested articulating members 38. Thus, small inputs to the proximal end 16 of the medical device 10 can result in large outputs at the distal end 14.

As shown here there are 18 independent pull wires 12. More or fewer pull wires 12 may be utilized without departing from the scope of the disclosure. The pull wires 12 may be stainless steel pull wires or formed of another high tensile strength material with low stretch characteristics such as DYNEEMA, KEVLAR, SPECTRA, and others. The pull wires 12 are attached to both the pull wire connector 46 and the support rings via a method that allows tension to be place on each wire independently and then locked in place simultaneously. The result of this operation is that all pull wires 12 are equally tensioned and all linear slop in the stack of components from the distal hub 42 to the support rings is eliminated prior to locking wires in place. The pull wires 12 may be attached via one or more of brazing, soldering, use of Loctite 680 cylindrical bond, welding, laser, sintering, resistance heating, screw clamps, etc.

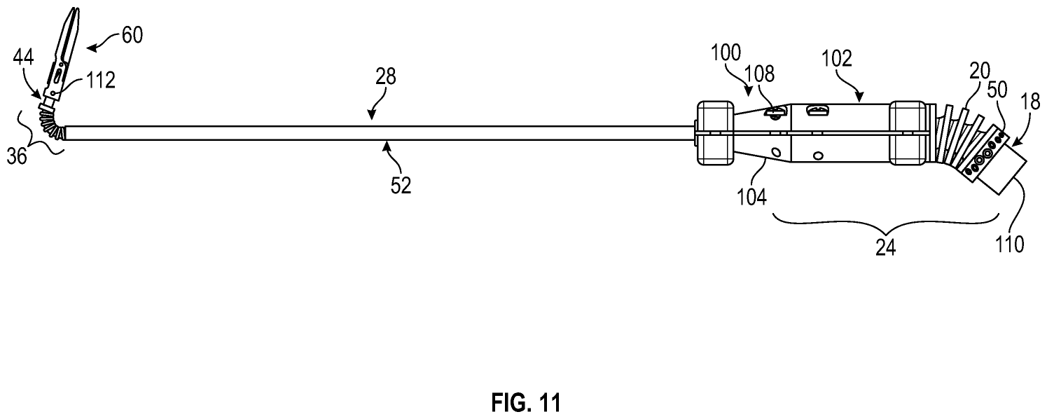

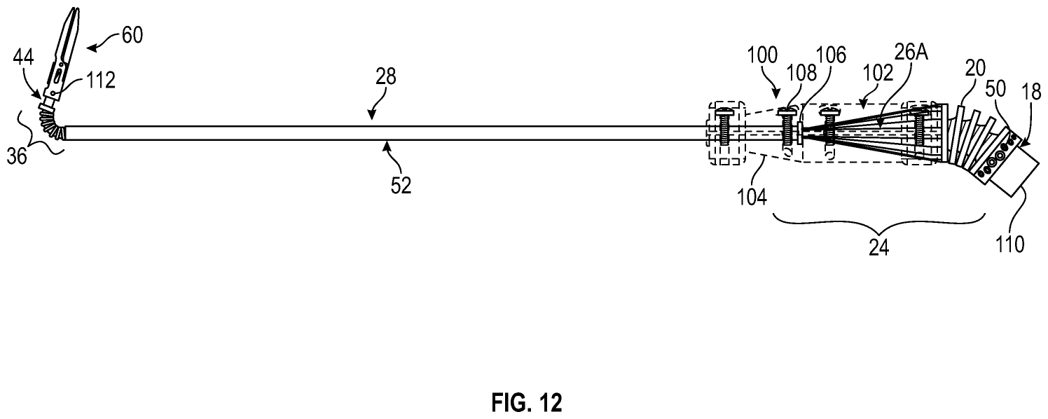

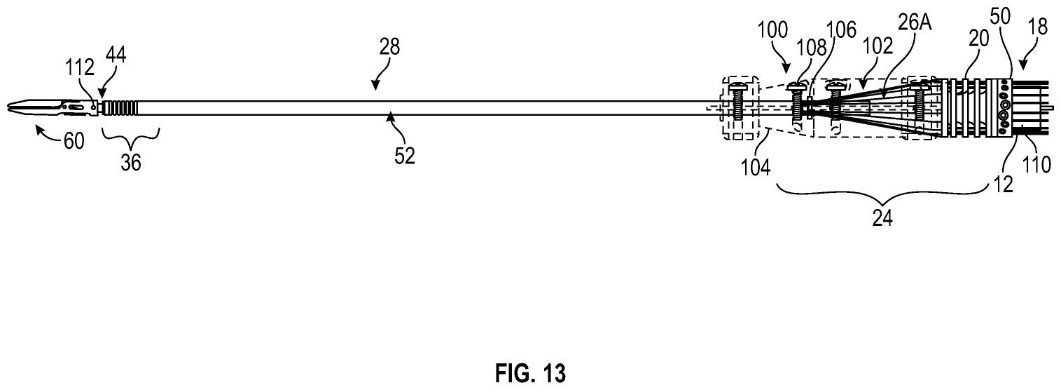

During assembly, the distal end of the pull wires 12 may be inserted into blind holes formed in the pull wire connector 46 and fixed in place under no tension at the time of fixation. At the proximal end, after threading of the pull wires 12 through the nested articulating members 38, rings 40, catheter 28 and its lumens 30, tapered hub 26 and its channels 27, articulating hubs 20 the proximal end of the pull wires are inserted into the support rings 18 with the pull wires extra-long to allow tension to be applied. Following application of tension to each of the pull wires 12, the pull wires are fixed in place, for example using a screw clamp 50, as depicted in FIGS. 11-13, or by brazing or any of the other methods described above. Following securement of the pull wires 12, the excess pull wire 12 is cut off. Where thermal techniques are employed to secure the pull wires 12 to the support ring 18, one of the support rings may be a thermal barrier to block the heat from melting the plastic components of the articulating hubs 20.

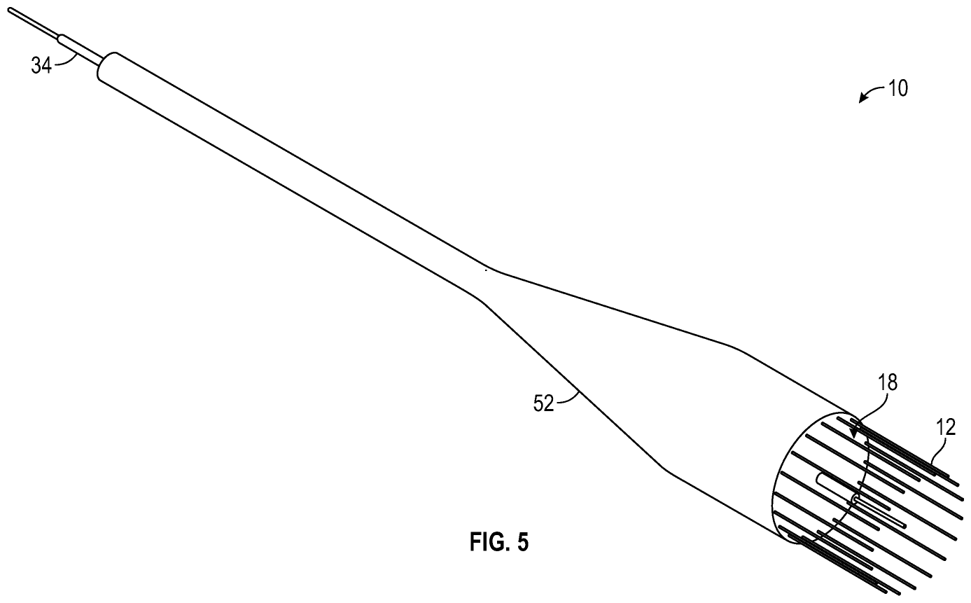

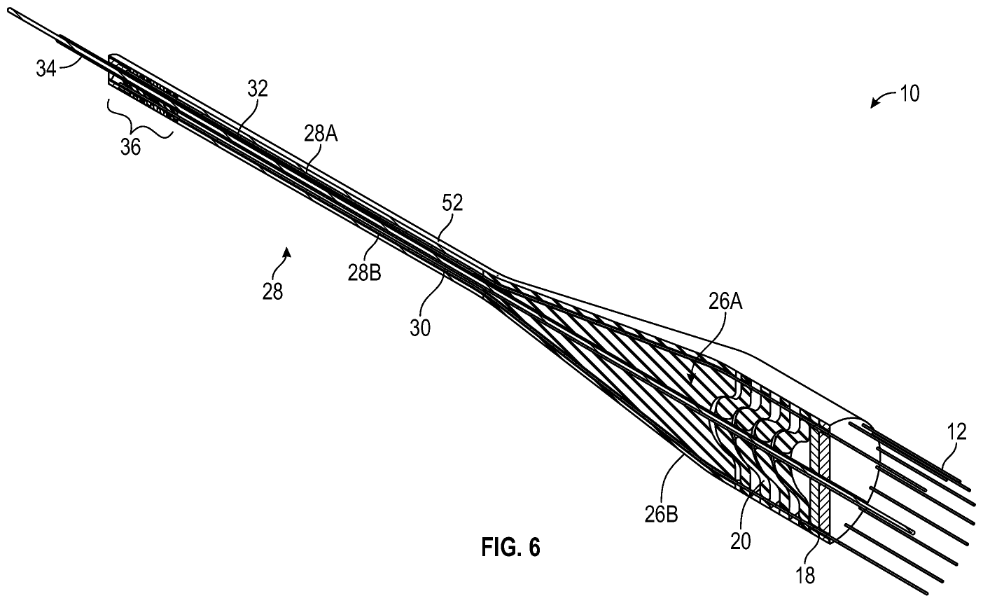

Following securement of the pull wires 12, a heat shrink sheath 52 as depicted in FIGS. 5 and 6 may be applied over the medical device 10. Application of heat to the heat shrink sheath 52 causes the heat shrink sheath 52 to reduce in diameter and conform to the shape of the medical device 10 and ensure that the pull wires 12 are protected. A second heat shrink sheath 54 may optionally be placed to protect the pull wires 12 at the point of connection to the pull wire connector 46.

In one embodiment of the disclosure, the diameter of the catheter 28 of the medical device 10 is approximately 3 mm and the bend radius of the distal articulating portion 36 is about 5.6 mm. However, other diameters of catheter including 5, 6, 7, 8, 9, 10 mm and others without departing from the scope of the disclosure as can different radii of ben of the distal articulating portion 36.

As noted above, the medical device 10 of the disclosure may include a tool actuator 34 for connection to a diagnostic tool or a therapeutic tool such as a forceps of an electro-surgical vessel sealer enable electro surgical procedures. FIG. 10 depicts a forceps 60 including a fixed jaw 62 and a movable jaw 64. The movable jaw 64 connects to tool actuator 34, and specifically a translating member 66 which is slidable relative to a fixed member 68. The fixed member 68 is secured relative to the fixed jaw 62 to place the two components in column with one another and prevent relative motion of the two components. The distal catheter 44 is secured to the fixed jaw 62, and may be received in the fixed jaw 62 in a securement region 70 via gluing, welding, brazing, or mechanical fastening such as swaging, or other methods known to those of skill in the art for connection of two components of a medical device. The translating member 66 of the tool actuator 34 connects to a pin 72 and rides in a slot 74 formed in the movable jaw 64. A second pin 76 is placed in an opening 78 formed in both the fixed jaw 62 and the movable jaw 64 to secure the fixed jaw 62 to the movable jaw 64. By advancement or retraction of the translating member 66 the pin 72 is translated in the slot 74. Advancement of the translating member 66 causes the movable jaw 64 to advance in the slot 74 and to open the movable jaw 64 relative to the fixed jaw 62 to place the jaws in the position depicted in FIG. 10. Retraction of the translating member 66 causes the pin 72 to translate distally in the slot 74 and to close the jaws.

Once the fixed jaw 62 and movable jaw 64 are proximate one another and subjected to suitable pressure, electrical current may be passed through the jaws from an electrical generator (not shown) to cause the proteins in the tissue to denature and coagulate. Such coagulation results in sealing of a blood vessel or other tissue placed between the jaws. Once so coagulated a knife (not shown) associated with forceps 60 may be advanced by a further manipulation of the tool actuator 34 to cut the coagulated tissue. Though described in detail in connection with a forceps 60, other forceps (e.g., having two movable jaws), and other end effectors, including vessel sealers, staplers, clip appliers, microwave and RF ablation antennae and others may be connected to the distal end 14 of the medical device and considered within the scope of the disclosure.

The medical device 10 may be connected at its proximal end 16 to a handle for manual manipulation by a clinician to open and close the forceps 60. Additionally or alternatively, the medical device 10 may be connected to a robotic arm to enable manipulation of the forceps 60 to achieve a desired position relative to the catheter 28. Further the robotic arm may be configured to open and close the forceps 60, seal tissue and advance a knife as described above.

With respect to electrosurgical aspects of the forceps, the forceps may be a monopolar arrangement and the translating member 66, may be energized by an electro surgical generator (not shown) and therewith energize the movable jaw 64. A pad (not shown) applied to a patient acts as the ground allowing for completion of the electrical circuit and the flow of energy through the body to achieve the vessel sealing. Alternatively, the forceps 60 may be a bipolar arrangement where the fixed jaw 62 is electrically connected, via for example the fixed member 68 and provide the ground path for the electrical energy to achieve coagulation of the tissue between the jaws.

Alternatively, in various embodiments one or more of the pull wires 12 may provide the electrical paths described above. Still further the knife (not shown) and knife drive mechanism (e.g., a cam rod, possibly made of Nitinol) which passes through the medical device 10 which terminates proximate the forceps 60 may act as the return path for the electrical current. As will be appreciated, the use of electrical energy is facilitated by the use of plastic materials in the catheters 28, articulating members 38, articulating hubs 20, etc., aid in electrically isolating the electrical paths provided by either the tool actuator 34 or of the pull wires 12 without fear of shorting the wires or causing any electrical shock to a user.

A further embodiment of the disclosure is depicted in FIGS. 11-13. In the embodiment of FIGS. 11-13 the heat shrink sheath 52 is limited to just the region of the catheters 28 between the distal articulating portion 36 and the proximal articulating portion 24. Proximate the joint between the catheters 28 and the tapered hub 26, a plastic clam shell 100, formed of an upper shell 102 and a lower shell 104 is employed to provide the outer cover portion 26B. In addition, a stop 106 is formed at the distal end of the inner portion 26A of the tapered hub 26. The stop 106 mates with recesses formed in the upper shell 102 and lower shell 104 to ensure that the inner portion 26A of the tapered hub 26 cannot move relative to the clam shell 100. Screws 108 connected the upper shell 102 with the lower shell 104 and create a clamping force that secures the catheter 28 in the clamshell. As with the cover portion 26B of the prior embodiment, the clam shell protects the pull wires 12 in the region where they are expanding from the diameter of the catheter 28 to the diameter of the articulating hubs 20. As shown in FIGS. 11-13, the pull wires 12 are secured to the support ring 18 via screw clamps 50.

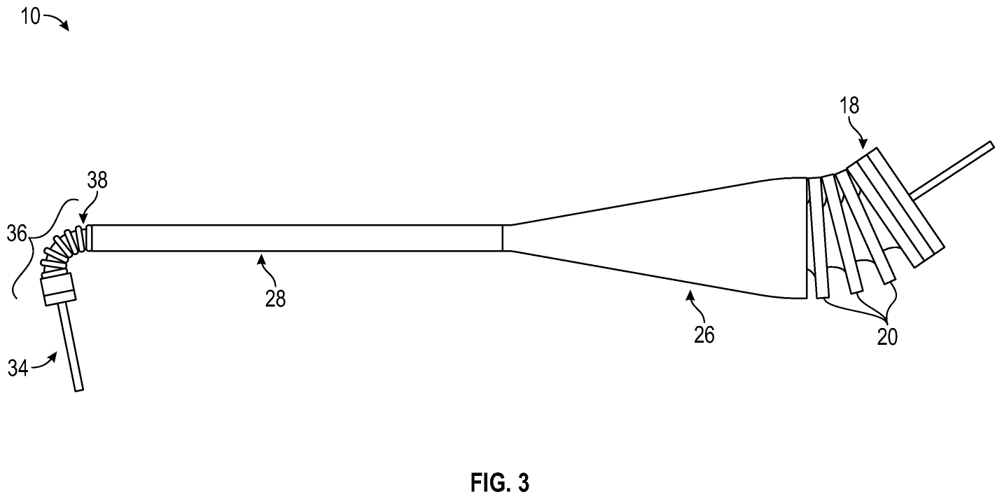

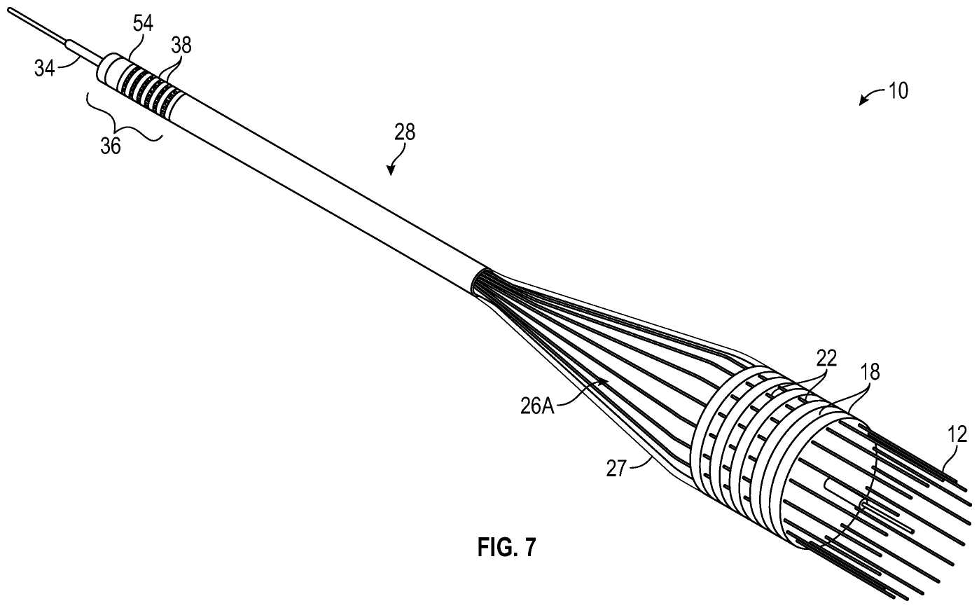

As shown in FIGS. 11-13 the support ring 18 includes a reduced diameter portion 110 which may be received in a handle or robot interface (not shown). On the distal end the forceps 60 may be secured to the distal catheter 44 via a pin 112. Other aspects of the embodiment of FIGS. 11-13 are substantially the same as those described in connection with FIGS. 1-10, and are not repeated here but any of the features of FIGS. 1-13 may be incorporated in any embodiment of the disclosure without departing from the scope of the disclosure.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects.

We claim:

1. An articulated medical device comprising:
a proximal articulating region including a plurality of articulating hubs;
a distal articulating region including a plurality articulating members;
at least one catheter extending between the proximal articulating region and the distal articulating region; and
a plurality of pull wires extending from the proximal articulating region to the distal articulating region and mechanically coupling and transferring movements of the proximal articulating region to the distal articulating region, wherein the movements of the proximal articulating region are amplified to a greater magnitude of movement in the distal articulating region.

2. The articulated medical device of claim 1, wherein the plurality of pull wires are equally tensioned.

3. The articulated medical device of claim 1 comprising, an inner catheter and an outer catheter, wherein the outer catheter defines a plurality of lumens configured for receiving the plurality of guide wires.

4. The articulated medical device of claim 3, wherein the inner catheter defines at least one lumen configured to receive a tool actuator.

5. The articulated medical device of claim 4, further comprising an end effector operably engaged with the tool actuator.

6. The articulated medical device of claim 5, wherein the end effector is a forceps.

7. The articulated medical device of claim 6, wherein the forceps is a bipolar electrosurgical vessel sealer.

8. The articulated medical device of claim 1, further comprising a tapered hub disposed between the plurality of articulating hubs and the catheter.

9. The articulated medical device of claim 8, wherein the tapered hub includes an inner portion including a plurality of channels configured for receiving the guide wires.

10. The articulated medical device of claim 1, further comprising at least one support ring.

11. The articulated medical device of claim 10, wherein the support ring is configured to receive and secure the plurality of pull wires.

12. The articulated medical device of claim 1, wherein the distal articulating region includes a plurality of pull wire connectors configured to secure the plurality of pull wires to the distal articulating region.

13. The articulated medical device of claim 12, further comprising a distal catheter configured to receive an end effector.

14. An articulated medical device comprising:
a proximal articulating region including a plurality of articulating hubs, a tapered hub, and at least one support ring;
at least one catheter extending from the tapered hub including a plurality of lumens formed therein;
a distal articulating region including a plurality articulating members, a distal catheter, and a plurality of pull wire connectors, wherein the distal articulating region is configured to mate with the at least one catheter; and
a plurality of pull wires secured the at least one support ring and extending from the at least one support ring, through the plurality of articulating hubs, in channels formed in the tapered hub, through the plurality of lumens formed in the at least one catheter, through the plurality of articulating members, and secured to the plurality of pull wire connectors, wherein the plurality of pull wires are equally tensioned along their length.

15. The articulated medical device of claim 14 further comprising, an inner catheter and an outer catheter, wherein the outer catheter defines the plurality of lumens configured for receiving the plurality of guide wires.

16. The articulated medical device of claim 15, wherein the inner catheter defines at least one lumen configured to receive a tool actuator.

17. The articulated medical device of claim 16, further comprising an end effector operably engaged with the tool actuator.

18. The articulated medical device of claim 17, wherein the end effector is a forceps.

19. The articulated medical device of claim 18, wherein the forceps is a bipolar electrosurgical vessel sealer.

20. The articulated medical device of claim 14, wherein movements of the proximal articulating region are amplified resulting in a greater magnitude of movement in the distal articulating region.
